(19) United States
(12) Patent Application Publication
(10) Pub. No.: US 2024/0090826 A1
(43) Pub. Date: Mar. 21, 2024

(54) SENSOR ARRANGEMENT, SYSTEM, AND METHOD FOR TISSUE ANALYSIS

(71) Applicants: Paul MEANEY, 412 96 (SE); Robin AUGUSTINE, Uppsala (SE); Syaiful Redzwan MOHD SHAH, Uppsala (SE); Mauricio David PÉREZ, Uppsala (SE); Taco BLOKHUIS, Bussum (NE)

(72) Inventors: Paul MEANEY, 412 96 (SE); Robin AUGUSTINE, Uppsala (SE); Syaiful Redzwan MOHD SHAH, Uppsala (SE); Mauricio David PÉREZ, Uppsala (SE); Taco BLOKHUIS, Bussum (NE)

(21) Appl. No.: 17/768,007

(22) PCT Filed: Oct. 14, 2020
(86) PCT No.: PCT/SE2020/050976

§ 371 Date: Apr. 11, 2022

(30) Foreign Application Priority Data
Oct. 14, 2019 (SE) 1951161-7

(51) Int. Cl. A61B 5/00; A61B 5/0507

(52) U.S. Cl. CPC A61B 5/4519; A61B 5/0507; A61B 5/4872; A61B 5/6832; A61B 5/7271; A61B 2560/02; A61B 2560/04; A61B 2562/0228

(57) ABSTRACT

The present invention relates to a sensor arrangement comprising a support substrate and at least two microwave antennas fixedly arranged in or on said support substrate, each microwave antenna being separated from all other microwave antennas by a band-stop structure configured to cancel direct coupling between the microwave antennas, said sensor arrangement further comprising microwave signal transmission paths configured to transmit microwave signals to or from said microwave antennas. The invention also relates to a system comprising such a sensor arrangement and a method for non-invasive assessment of a property of subdermal tissue in a subject.

### FIELD OF THE INVENTION

The present invention relates to the field of analysis of biological tissues, and in particular microwave based techniques to analyze variations in biological tissue considering the effect of physiological and biological properties on microwave signals.

### BACKGROUND

Low muscle mass is associated with negative outcomes such as more surgical complications, longer length of hospital stays, lower physical function, poorer quality of life and shorter survival. As such, the potential clinical benefits of preventing and reversing this condition for are likely to impact patient outcomes and resource utilization/health care costs along the care continuum. In secondary care such as geriatric care and home care, quality of life in ageing people is hampered by a decline in muscle mass, with an increased risk of falling, increased morbidity in disease and a decreased life expectancy.

This decline in muscle mass in combination with strength and function, a condition known as sarcopenia, is recognized in many medical conditions as an important risk factor for mortality and morbidity. The significance of sarcopenia is evident in a variety of diseases, in various patient groups and in different clinical settings, and therefore across the whole continuum of care. Sarcopenia can be prevented or treated by physical activity and nutritional intervention. Although these interventions have been shown to be cost-effective, the condition has to be diagnosed before any treatment can be initiated and continuously optimized depending on patients' body composition and treatment profile. A delay in detection of sarcopenia increases the risk of falling, hampers mobility, increases the risk of severe complications in people undergoing treatment for diseases, and has a negative influence on survival. In the healthcare system, the treating physicians as well as the healthcare system itself are affected because of the higher complication rates and lower survival of patients. The associated longer admission times and higher costs put an additional burden on the system.

In several widely adopted definitions, bioimpedance, CT scanning, and muscle function determined by mobility tests or strength tests are all used. None of these tests shows high accuracy or reproducibility, leading to underdiagnosis of sarcopenia. Moreover, the availability of techniques like CT scanning hampers their widespread use and makes repeated measurements at regular intervals, for example to monitor the effect of interventions and ongoing treatment, impossible. As a consequence, people either are either not identified as sarcopenic, or the effect of treatment cannot be evaluated.

Currently the assessment of body composition is done using CT, MRJ and DEXA scans. Ultra sound and Body Impedance Analysis (BIA) are other techniques. All these methods suffer from high cost constrains as far as regular patient follow-ups are concerned. Some of them are not even suited for frequent follow-ups due to radiation concerns. Others such as BIA and Ultrasound suffer from lack of resolution and accuracy. Especially in the case of BIA body fluids (electrolyte) play a decisive role in the analysis outcome, which means the patient's water saturation level can alter the body composition readings. This is a major shortcoming of BIA technique.

Human body communications (HBC), also termed IntraBody Communication (IBC), have lately received a good deal of interest in healthcare monitoring and treatment applications.

Wearable sensor arrangements comprising microwave antennas are known in the art, such as through US2014/0253397 and US2018/0042513. Sensor arrangements comprising microwave antennas for introduction into the body are also known in the art, such as through WO2018/081602.

### SUMMARY

The present invention aims to provide a new diagnostic and multimodal approach for measurement of body composition that is simple, accurate, non-ionizing, and readily available for the benefit of people at risk for low muscle mass resulting in sarcopenia across a large spectrum of the general population.

While the sensor arrangements according to the prior art may function well for their intended use, they are not configured for use according to the present invention. In particular, the present invention relates on analysis of signals transmitted between at least two microwave antennas within the tissues of a subject. It is therefore necessary to cancel direct coupling between the antennas through creeping wave coupling on the body surface. This is a non trivial task due to the off-the plane propagation initiated by the fringe fields around the antennas. This problem is solved by the present inventors by inclusion of a band-stop structure that cancel both in-plane and off-plane signal propagation, i.e. cancel direct coupling between the microwave antennas.

According to a first aspect there is provided a sensor arrangement comprising a support substrate and at least two microwave antennas fixedly arranged in or on the support substrate, each microwave antenna being separated from all other microwave antennas by a band-stop structure configured to cancel direct coupling between the microwave antennas, the sensor arrangement further comprising microwave signal transmission paths configured to transmit microwave signals to or from the microwave antennas.

The sensor arrangement according to the invention provides a well-defined sensor set-up that reduces disturbances caused by movement of the antennas and direct coupling between the antennas.

According to some embodiments, said microwave antennas are fixedly arranged 20-160 mm apart, such as 20, 30, 40, 50, 60, 70, 80, 90, 100, 110, 120, 130, 140, or 150 mm apart.

According to some embodiments, said microwave antennas are split ring resonators.

According to some embodiments, the sensor comprises one or more microwave signal contacts connected to the transmission paths to the microwave antennas to facilitate easy connection to, and disconnection from, a microwave signal generator and/or a microwave signal processing unit.

According to some embodiments, the sensor comprises at least one microwave switch arranged in a transmission path between a microwave signal contact and two or more microwave antennas.

According to some embodiments, at least three microwave antennas are fixedly arranged in or on said support substrate.

This enables simultaneous collection of signal data from a plurality of receiver antennas and provides for a more extensive analysis of the biological tissue.

According to some embodiments, the support substrate is flexible.

The sensor arrangement needs good contact with skin surface for efficient signal coupling. Selection of substrate material should thus preferably make the solid substrate flexible and/or stretchable to provide good contact when applied on a body of a subject. Polydimethysiloxane (PDMS) and Galinstan (Liquid metal) based stretchable fabrication technology are currently contemplated to ensure surface compliance.

According to some embodiments, the support substrate has an adhesive coating for fixing the sensor arrangement on the skin of a subject.

According to a second aspect there is provided a system comprising a sensor arrangement according to the first aspect; a tuneable microwave signal generator configured to generate and transmit a microwave signal to a first microwave antenna in the sensor arrangement; and a signal processing unit arranged to receive the transmitted microwave signal from at least one second microwave antenna in the sensor arrangement.

According to some embodiments, the signal processing unit is configured to analyse a change in the transmitted microwave signal between said first microwave antenna and said at least one second microwave antenna.

According to some embodiments, the change in said microwave signal is a change in amplitude and/or a phase delay.

According to some embodiments, the tuneable microwave signal generator can generate microwave signals in a frequency range of 2.45 GHz to 10 GHz.

According to some embodiments, the system is suitable for non-invasive assessment of a property of subdermal tissue in a subject, and the signal processing unit is configured to correlate a change in the transmitted microwave signal between the first microwave antenna and the at least one second microwave antenna with properties of subdermal tissue in the subject.

According to some embodiments, the sensor arrangement incorporated in the system comprises at least three microwave antennas and the signal processing unit is arranged to receive said microwave signal from at least two second microwave antennas in said sensor arrangement; and the signal processing unit is configured to correlate a change in the transmitted microwave signal between the first microwave antenna and each of the at least two second microwave antennas with the property of subdermal tissue in a subject.

According to some embodiments, the property of subdermal tissue is selected from the group consisting of fat content of muscle tissue, thickness of muscle tissue, thickness of fat tissue.

According to a third aspect there is provided a method for non-invasive assessment of a property of subdermal tissue in a subject comprising the steps
S1. Providing a first and at least one second microwave antennas in connection with the skin of the subject, each microwave antenna being separated from all other microwave antennas by a band-stop structure configured to cancel direct coupling between the microwave antennas and provided at a fixed distance from all other microwave antennas;
S2. Transmitting a microwave signal from the first microwave antenna;
S3. Receiving the microwave signal at the at least one second microwave antenna after propagation through subdermal tissue of the subject;
S4. Optionally repeating steps S2 and S3 with a microwave signal of a different frequency; and
S5. Correlating a change in the transmitted microwave signal or signals between the first microwave antenna and the at least one second microwave antenna with properties of subdermal tissue in the subject.

According to some embodiments, the method comprises the steps
S1. Providing a first and at least two second microwave antennas in connection with the skin of said subject, each microwave antenna being separated from all other microwave antennas by a band-stop structure configured to cancel direct coupling between the microwave antennas and provided at a fixed distance from all other microwave antennas;
S2. Transmitting a microwave signal from the first microwave antenna;
S3. Receiving the microwave signal at each of the at least two second microwave antennas after propagation through subdermal tissue of the subject;
S4. Optionally repeating steps S2 and S3 with a microwave signal of a different frequency; and
S5. Correlating a change in the transmitted microwave signal or signals between the first microwave antenna and each of the at least two second microwave antennas with properties of subdermal tissue in the subject.

According to some embodiments, the change in the transmitted microwave signal is a change in amplitude and/or a phase delay.

According to some embodiments, steps S2 and S3 are repeated at least four times with microwave signals of frequency 2.45; 5, 8, and 10 GHz.

According to some embodiments, the property of subdermal tissue is selected from the group consisting of fat content of muscle tissue, thickness of muscle tissue, thickness of fat tissue.

Effects and features of the second and third aspects are to a large extent analogous to those described above in connection with the first aspect. Embodiments mentioned in relation to the first aspect are largely compatible with the second and third aspects.

The present disclosure will become apparent from the detailed description given below. The detailed description and specific examples disclose preferred embodiments of the disclosure by way of illustration only. Those skilled in the art understand from guidance in the detailed description that changes and modifications may be made within the scope of the disclosure.

Hence, it is to be understood that the herein disclosed disclosure is not limited to the particular component parts of the device described or steps of the methods described since such device and method may vary. It is also to be understood that the terminology used herein is for purpose of describing particular embodiments only, and is not intended to be limiting. It should be noted that, as used in the specification and the appended claim, the articles “a”, “an”, “the”, and “said” are intended to mean that there are one or more of the elements unless the context explicitly dictates otherwise. Thus, for example, reference to “a unit” or “the unit” may include several devices, and the like. Furthermore, the words “comprising”, “including”, “containing” and similar wordings does not exclude other elements or steps.

### BRIEF DESCRIPTION OF THE DRAWINGS

The above objects, as well as additional objects, features and advantages of the present disclosure, will be more fully appreciated by reference to the following illustrative and non-limiting detailed description of example embodiments of the present disclosure, when taken in conjunction with the accompanying drawings.

FIG. 1 shows a schematic illustration of a sensor arrangement according to (A) an embodiment comprising two microwave antennas, and (B) three microwave antennas.

FIG. 2 is a perspective view of a Split Ring Resonator useful as a microwave antenna in the present invention.

FIG. 3 shows a schematic illustration of a system according to the invention.

FIG. 4 shows a schematic illustration of a sensor arrangement in use on biological tissue.

FIG. 5 is a flow chart illustrating a method according to the invention.

FIG. 6 shows an arrangement used to characterize the EM signal loss on biological tissues in a numerical simulation analysis.

FIG. 7: (A) The Simulated reflection coefficient, S₁₁; (B) The Simulated signal coupling, S₂₁ between the two SRRs at the distance 20 mm for two scenarios of free space and body tissue channel.

FIG. 8: The comparison of (A) Amplitude of S₂₁ and (B) Phase of S₂₁ when the thickness of skin=2.5 mm, fat=20 and 35 mm (T), and muscle 50 mm. The SRR sensors placed on top of the skin layer with gap distance from 20 mm to 160 mm (D).

FIG. 9: The simulated and measured S₂₁ results when the thickness of skin=2.5 mm, muscle thickness varied from 10 mm to 50 mm in 20 mm step, fat layer varied from 5 mm to 35 mm by 10 mm step. The SRR sensors placed on top of the skin layer and varies with (a) Distance=0 mm (b) Distance=50 mm (c) Distance=100 mm (d) Distance=150 mm (e) Distance=200 mm and (f) Distance=250 mm

FIG. 10: The measured and simulated (within parantheses) S₂₁ results when the thickness of skin=2.5 mm, (a) Fat=5 mm, (b) Fat=15 mm, (c) Fat=25 mm and (d) Fat=35 mm, respectively for muscle thickness varied from 10 mm to 50 mm in 20 mm step. The SRR sensors were placed on top of the skin layer and varied with distance from 0 to 250 mm.

FIG. 11: The E-field distribution depending on the thickness of the tissue (a) Fat=5 mm (b) Fat=25 mm and (c) Fat=35 mm at 100 mm distance of SRR sensors.

FIG. 12: The E-field penetration depending on the thickness and variation of distance. (a) Fat 5 mm, Muscle 10 mm, 30 mm, 50 mm (skinny condition), (b) Fat 25 mm, Muscle 10 mm, 30 mm, 50 mm (normal condition) and (c) Fat 35 mm, Muscle 10 mm, 30 mm, 50 mm (high-fat condition).

FIG. 13 is an illustration of microwave signal confinement in different tissues with respect to the frequency of operation.

### DETAILED DESCRIPTION

The present invention uses the Radio Frequency (RF) propagation technique to understand the geometrical distribution of a multi-layered tissue by calculating the signal loss while the signal is propagating through the tissues.

The received signal's signal-to-noise ratio (SNR), which requires the underlying multilayer human body that governs the microwave propagation, and its impact on attenuation must be resolved so that Human Body Communication (HBC) systems are designed and implemented accurately. One of the main considerations of these requirements is the study of electromagnetic (EM) signal propagation characteristics in the body tissue. Microwave propagation is then investigated based on the tissue dielectric properties in terms of their reflection, signal loss, attenuation, and penetration depth.

In fat channel microwave communication, the human fat tissue acts as the main signal propagation medium, which implies that the dimensional variations in the fat channel will be reflected in the signal coupling level. This attribute opens up a sensing possibility of the physical dimensions of the channel, which is essentially the thickness of fat tissue. The fat channel is confined by skin and muscle tissues. While variations in the skin thickness are not significant, variations in the muscle could be. In other words, both fat and muscle thickness variations can have a combined effect on fat channel communication. In this work, we utilize the variability between different tissue thicknesses as a precursor to assess the signal attenuation level, which in turn, is a marker of underlying tissue distribution.

The technique for tissue analysis according to the invention is non-invasive, non-ionizing and objective compared to other state-of-the-art modalities.

In one aspect, the present invention relates to a device (a sensor arrangement) comprising two antennas separated by a fixed distance, of which one sends interrogating signals to assess the tissue properties whereas the other antenna serves as the receiver. The antennas are separated by a physical structure (a band stop structure) that obstructs the formation of creeping waves resulting in direct signal coupling between the antennas. Instead, coupling between the antennas are provided for deeper within the tissues. Furthermore the frequency of operation of the antennas may be tuned to increase the penetration of the microwave signal into the tissue, controllable through different wavelength's angle of incidence behaviour to select any particular subdermal tissue for interrogation.

The feasibility of the concept mentioned above is examined using microstrip Split Ring Resonators (SRRs) to estimate the EM signal loss through biological tissue. Two prototypes consisting of three layers of tissue thickness (skin, fat and muscle) are presented primarily for the measuring conditions and the personal characteristics of human tissues.

This invention indicates an analysis approach to examine the influence of the tissue proportions on the EM signal coupling.

In the exemplary section, a laboratory setup comprising of two SRR sensors and an ex-vivo porcine experimental model for biological tissues are disclosed. Also provided is an intensive parametric analysis of a variety of fat and muscle thickness values at different sensor distances, which enabled us to conclude the underlying EM signal coupling. Finally, a validation between electric field (Efield) and penetration depth and their associated effects on signal loss due to the variation in thickness and distance is provided.

The present disclosure will now be described with reference to the accompanying drawings, in which preferred example embodiments of the disclosure are shown. The invention may, however, be embodied in other forms within the scope of the appended claims and should not be construed as limited to the herein disclosed embodiments.

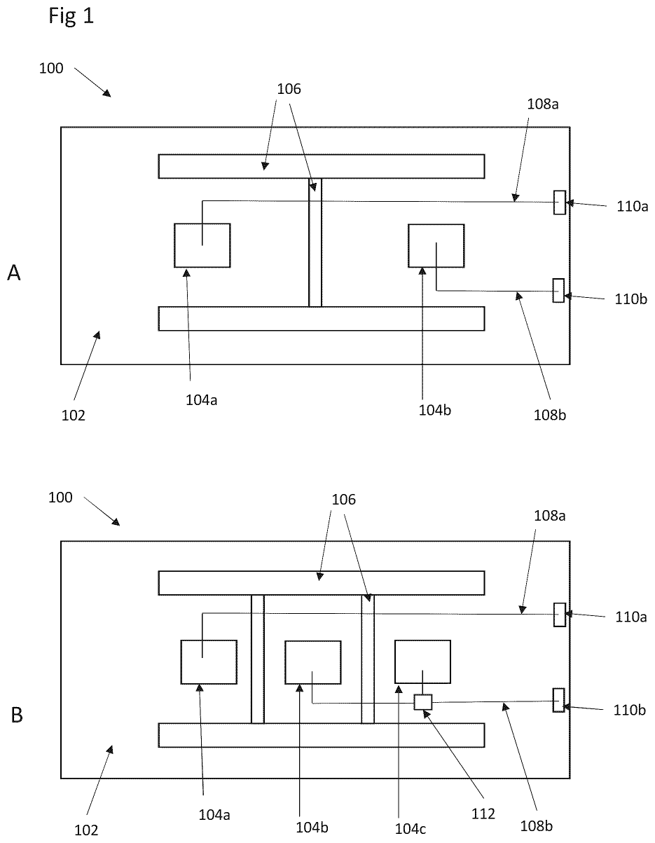

FIG. 1A shows a sensor arrangement 100 comprising a support substrate 102 and two microwave antennas 104a, 104b fixedly arranged in or on the support substrate 102, each microwave antenna 104a, 104b being separated from all other microwave antennas 104 by a band-stop structure 106 configured to cancel direct coupling between the microwave antennas 104, the sensor arrangement 100 further comprising microwave signal transmission paths 108 configured to transmit microwave signals to or from the microwave antennas 104. Two microwave signal contacts 110a, 110b connected to the transmission paths 108 to the microwave antennas 104 are provided for connection of the sensor arrangement to a system according to the invention.

FIG. 1B shows a sensor arrangement 100 comprising a support substrate 102 and three microwave antennas 104a, 104b, 104c fixedly arranged in or on the support substrate 102. FIG. 1B also shows a microwave switch 112 arranged in a transmission path between a microwave signal contact 110 and two microwave antennas 104b, 104c.

The microwave antennas 104 may be fixedly arranged 20-250 mm, such as 20-160 mm apart. In embodiments having more than two microwave antennas, the microwave antennas being separated by the longest distance may be separated by up to 160 or 250 mm, and the other microwave antenna(s) may be evenly distanced from the longest separated microwave antennas, or by fixed distances such as 20, 30, 40 or 50 mm.

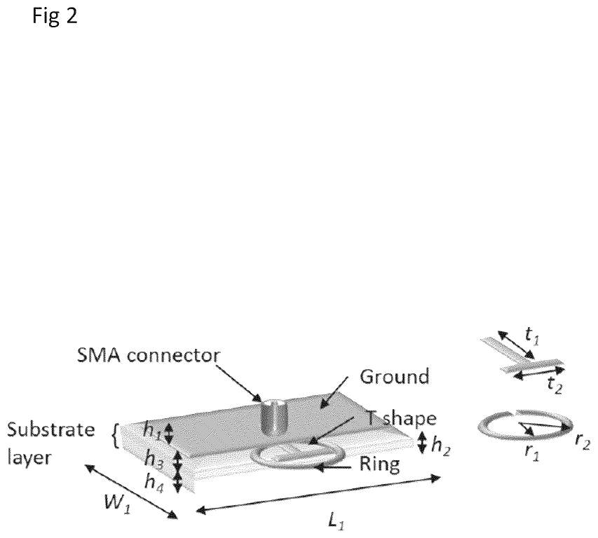

In embodiments, said microwave antennas are split ring resonators. The split ring resonator sensor design used the concept of a single split microstrip ring resonator (known as microstrip gap) as illustrated in FIG. 2. Model parameters used to develop the split ring resonator are shown in Table 1.We used three different layers of the substrate to design the split ring resonator sensors. Layer 1 is ground plane with the thickness h1=0.035 mm. Layer 2 with the thickness h2 is made of TMM4 substrate (εᵣ=4.5, loss tangent, tan δ=0.002) with thickness h2=0.635 mm and layer 3, h3 is from TMM6 substrate (εᵣ=6, loss tangent, tan δ=0.0023) with thickness h3=0.635 mm. Both layers 2 and 3 are fabricated using TMM high-frequency laminates. In addition, layer 4 with the thickness of h4 is fabricated using Rogers 6010 substrate (εᵣ=10.2, loss tangent, tan δ=0.0023) with thickness h4=0.635 mm, forming a total thickness of about 1.905 mm. In the lab prototype, all the layers were stacked together using adhesive glue. Specifically for layer 4, we used this as a matching layer is principally made up of a substrate sheet, which performs as a coupling material to the target (which is skin tissue) and makes it possible to radiate more EM waves into the biological tissues and achieve stable resonance frequency while illuminating the targets. The structure of the SRR has been proved to have a penetration between 10 and 15 mm when applied on the surface of the skin in distal and thigh positions (upper part of the leg —femur) as shown by Redzwan et al. [S. R Mohd Shah et al., Sensors (Basel, Switzerland), vol. 18, no. 2, pp. 636, Feb 2018] on human volunteers.

Each tissue is characterized by differences in dielectric properties, focusing primarily on relative permittivity, εᵣ, and conductivity, σ. In particular, the conductivity of skin and muscle tissues at high frequencies is much higher than the conductivity of fat tissue. This is because of the high water content in skin and muscle compared to the low content of water in fat and bone.

The present invention is complementary to this approach. The signal connection was chosen to be perpendicular to the ring's plane and at the center of the ring's projection on the ground plane. Therefore, a SubMiniature version A (SMA) connector was employed and the signal's transition to a microstrip line starts from the bottom (ground) in the center of the ring's projection and to the edges of the parallel section of T-shape microstrip line. The sensor input impedance is optimized to be close to 50.

TABLE 1
Model Parameters used for investigating signal
transmission in biological tissues.

| Categories | Type | Label | Dimension (mm) |
|---|---|---|---|
| Split Ring | Ground | h₁ | 0.035 |
| Resonator | Substrate | W₁ | 25 |
| | | L₁ | 25 |

| | | |
|---|---|---|
| h₂ | 0.635 | (TMM4) |
| h₃ | 0.635 | (TMM6) |
| h₄ | 0.635 | (Roger 6010) |

| | | | |
|---|---|---|---|
| | Ring Resonator | r₁ | 8.6 |
| | | r₂ | 5.8 |
| | T-shape | t₁ | 9.2 |
| | | t₂ | 4.2 |
| Tissues | Skin | h₅ | 2.3 |
| | Fat | h₆ | 5-35 |
| | Muscle | h₇ | 10-50 |
| | Width | W₂ | 120 |
| | Length | L₂ | 250 |

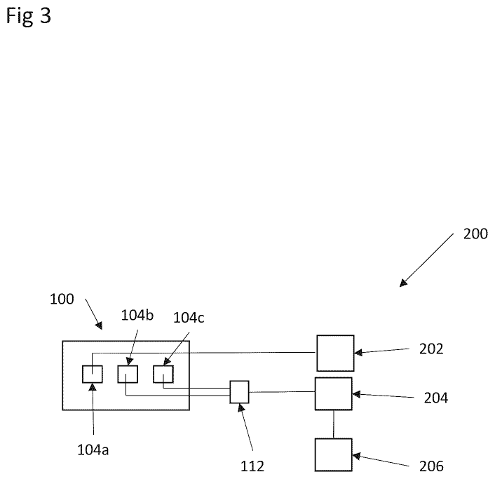

As shown in FIG. 3 the system according the second aspect of the invention comprises a transmitter probe (104a) and a pair of primary (104b) and secondary (104c) receiver probes fixed at specified distances. The system further comprises a tuneable signal generator (202), a signal processing unit (204) and optionally a switch (112) incorporated in the sensor arrangement 100 or between the sensor arrangement and the signal processing unit (204). In some embodiments, the tuneable microwave signal generator can generate microwave signals in a frequency range of 2.45 GHz to 10 GHz. The system (200) may further comprise one or more communication units (206) for communicating a result produced by the signal processing unit (204) to a user of the system or to a data storage or other system. The communication unit (206) could thus be a display, and/or a wired or wireless link to a data storage which may be accessed by the user. Some details of the sensor arrangement included in the system 200 have been omitted in FIG. 3 for clarity, but are disclosed in FIGS. 1A and 1B. The positioning of the switch may be as in FIG. 1B or as in FIG. 3, or as otherwise deemed functional.

The signal processing unit 204 is configured to analyse a change in the transmitted microwave signal between said first microwave antenna 104a and said at least one second microwave antenna 104b. The signal processing unit (204) preferably has enough computational capacity to process the received signals and calculate the respective properties of subdermal tissue of the patient in real-time. The provision of a communication link to a data storage will help the data to be logged and stored in the data storage, such as a cloud service, and thus help to make a good estimate of patient progress in rehabilitatory settings. Thus the system (200) will be a versatile system offering real-time insight into the body composition and at the same time registering the variations of muscle mass over time which ensures care continuum.

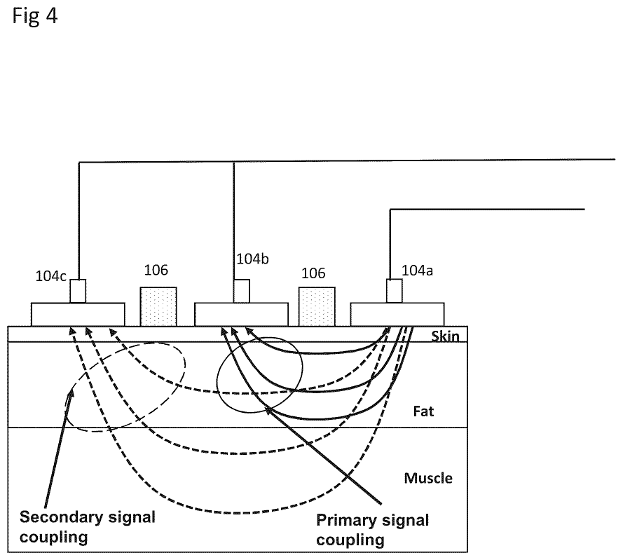

FIG. 4 shows the sensor arrangement applied to the skin of a subject. The signal from the transmitter probe (104a) will reach the primary probe (104b) by direct coupling through coupling through the fat tissue and by reflection from the muscle tissue. The secondary probe (104c) receives mostly the signals through fat tissue and muscle tissue, and to some part by reflection from muscle tissue. The combinations of received signals have already traversed through different tissues and have subjected to phase delay and signal absorption.

Deducing the signal amplitude and phase delay at receiver probes (104b, 104c) provides the information on tissue composition. The microwave signal generator (202) is configured to generate different signal frequencies enabling the signal confinement in various tissues as shown in the FIG. 3. This provides for accurate quantification of subdermal tissues such as fat and muscle.

To avoid signal leakage into the free-space (air) from the transmitter probe (104a), which would limit the signals from coupling into deeper tissue layer, band-stop structures (106) as shown in FIGS. 1 and 4 are provided between the transmitter probe (104a) and receiver probes (104b, 104c). Band-stop structures will obstruct the lateral passage of Microwave/Radio frequency (RF), that would otherwise result in direct signal coupling between probes. The band-stop structures (106) should thus in one embodiment be able to cancel both in-plane and off-plane signal propagation, such as cancel out the mutual coupling from 0-45 degree angle with respect to the plane of the transmitter and receiver probes. It will also help in focusing the signal deeper into the tissue at the minimum power available. The inherent coupling between the probes may however be good enough to estimate tissue composition with required accuracy, even if the band-stop structures do not completely abolish direct signal coupling between probes.

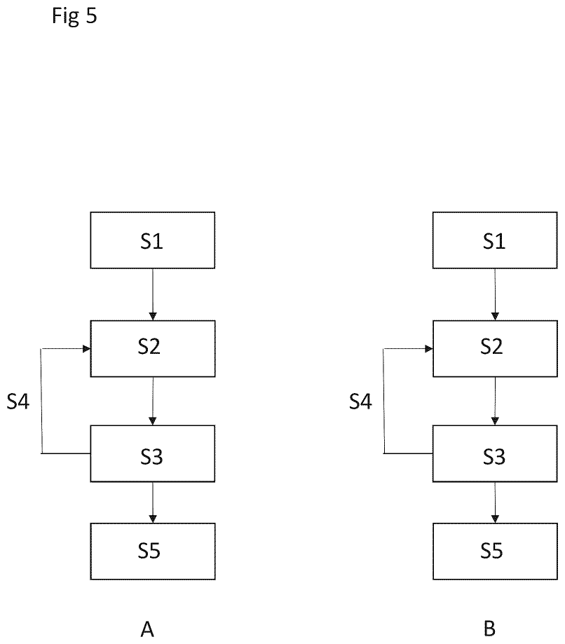

The third aspect of this disclosure shows a method for non-invasive assessment of a property of subdermal tissue in a subject as illustrated in FIG. 5. FIG. 5A shows a flow chart for such method comprising the steps
S1. Providing a first and at least one second microwave antennas in connection with the skin of the subject, each microwave antenna being separated from all other microwave antennas by surfaces having an impedance sufficient to cancel direct coupling between the microwave antennas;
S2. Transmitting a microwave signal from the first microwave antenna;
S3. Receiving the microwave signal at the at least one second microwave antenna after propagation through subdermal tissue of the subject;
S4. Optionally repeating steps S2 and S3 with a microwave signal of a different frequency; and
S5. Correlating a change in the transmitted microwave signal or signals between the first microwave antenna and the at least one second microwave antenna with properties of subdermal tissue in the subject.

FIG. 5B shows a flow chart for such method comprising the steps
S1. Providing a first and at least two second microwave antennas in connection with the skin of said subject, each microwave antenna being separated from all other microwave antennas by surfaces having an impedance sufficient to cancel direct coupling between the microwave antennas;
S2. Transmitting a microwave signal from the first microwave antenna;
S3. Receiving the microwave signal at each of the at least two second microwave antennas after propagation through subdermal tissue of the subject;
S4. Optionally repeating steps S2 and S3 with a microwave signal of a different frequency; and
S5. Correlating a change in the transmitted microwave signal or signals between the first microwave antenna and each of the at least two second microwave antennas with properties of subdermal tissue in the subject.

In embodiments, the change in the transmitted microwave signal is a change in amplitude and/or a phase delay.

In embodiments, steps S2 and S3 are repeated a plurality of times with different frequencies, such as repeated 1, 2, 3, 4, 5, 6, 7, 8, 9, or 10 times or more. Each repetition may be performed with microwave signals of frequency in a range from 2 GHz, 2.45 GHz, 5 GHz, 8 GHz, or 10 GHz, to 2.45 GHz, 5 GHz, 8 GHz, or 10 GHz. In one embodiment steps S2 and S3 are repeated at least four times with microwave signals of frequency 2.45; 5, 8, and 10 GHz.

In embodiments, the property of subdermal tissue is selected from the group consisting of fat content of muscle tissue, thickness of muscle tissue, thickness of fat tissue.

In some embodiments, signal loss or path loss for the transmitted microwave signal or signals between the first microwave antenna and each of the at least two second microwave antennas is correlated with properties of muscle tissue in the subject.

In some embodiments, the correlation is performed by comparing the observed change, such as the observed signal loss or path loss, with a look-up table created by recording observed changes in the microwave signal for a number of biological tissue samples of known properties, optionally at a plurality of different frequencies.

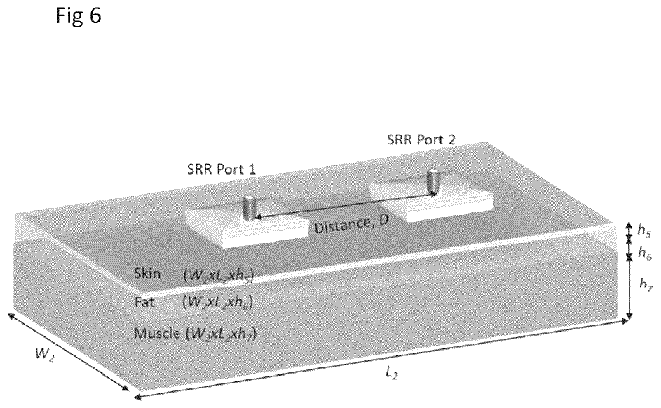

For purpose of numerical simulation analysis of signal loss, we show the development of two 3D models using the Computer Simulation Technology (CST) software based on Ultra Sound (US) tissue thickness measurements. FIG. 6 demonstrates the suggested arrangement that is used to characterize the EM signal loss on the biological tissues. To study the interaction of the proposed sensors and ex-vivo model, a transmitter sensor (Tx) is placed to create an EM signal that perpendicularly propagates into multilayer tissues; meanwhile, a receiver sensor (Rx) performs to identify the received EM signal. The geometrical thickness of every tissue layer, which includes the numerical and experimental ex-vivo, is listed in Table 1. Biological tissue properties included in the simulation are applied at a frequency of 2.45 GHz to excite the behavior of three tissue layers depending on the thickness tissue.

The inventors' approach is to develop a multilayer homogeneous model that have been considered for numerical and experimental studies. This model consists of a three-layer tissue thickness containing skin, h₅, fat, h₆ and muscle, h₇. Here, the skin thickness is kept constant (2.3 mm), while, the fat and muscle thicknesses are varied from 5 mm to 35 mm in 10 mm steps and from 10 mm to 50 mm in 20 mm steps, respectively. The length of simulation model is 250 mm and the width is 120 mm, which optimizes the condition for signal loss. As mentioned earlier, human tissues can be classified into those with high water content (like muscle and skin) and those with low water content (like fat).

Therefore, the influences of fat and muscle thickness on the EM signal loss between transmitter and receiver sensor are examined and analyzed. FIG. 6 illustrates the three-layered model structure that used to characterize the influence of EM signal loss. A transmitter sensor generates an EM signal perpendicularly propagating through the other upper tissue layer, while a receiver sensor is used to detect the received signal by varying the distance between them from 0 mm to 250 mm in 50 mm steps.

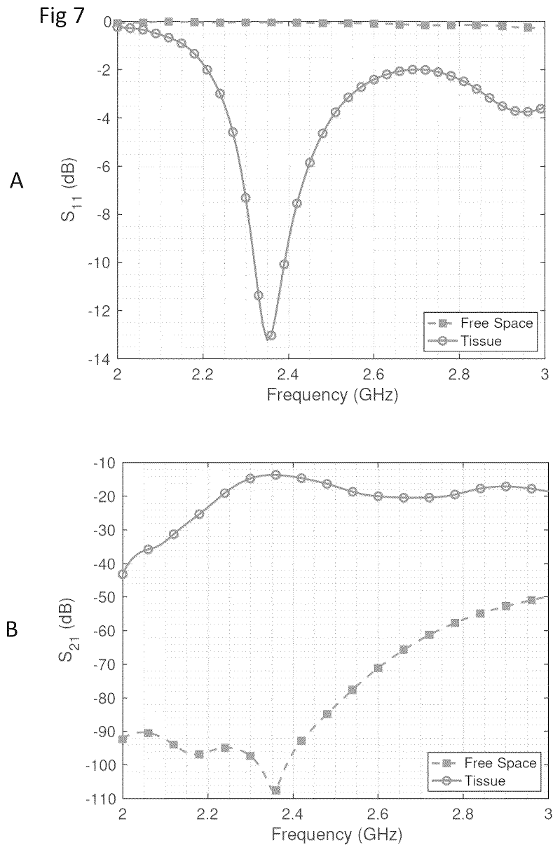

A numerical study was performed to demonstrate that an SRR has the sensing capability with which multilayer tissues can be analyzed. The SRRs are placed above the body tissue, which is a multilayer medium consisting of air, skin, fat and muscle, to couple the EM signal inside the body tissue. The distance between the two SRRs are varied from 20 mm to 160 mm, and the amplitude and phase of the coupled signal for the fat layer are reported. As shown in FIG. 7A, the SRRs do not resonate in the free space; however, when the SRR is located over the body tissue, it resonates at the central frequency of 2.35 GHz. So, the SRR is sensitive to the characteristic parameters of the body tissue; as soon as the tissue parameters vary, a significant variation in the resonance frequency of the resonator occurs.

To calculate the free space coupling of the SRRs, two simulations are conducted. In the first simulation, the two SRRs are apart from each other and are connected with free space channel and in the second simulation; they are placed on the body tissue. FIG. 7B shows the coupling between the two SRRs, and for the case of free space, it is observed that at the resonance frequency of 2.35 GHz the coupling through free space is below −90 dB and the coupling through body tissue is −14 dB. Therefore, it is clear that the SRR has the potential to couple the EM signal inside the fat layer compared to the coupling through the free space. The entire signal coupling is done through the body channel. With the increase in the distance of the two SRRs, there will be an increase in the path loss. However, the path loss is nonlinear because the system is a near-field system.

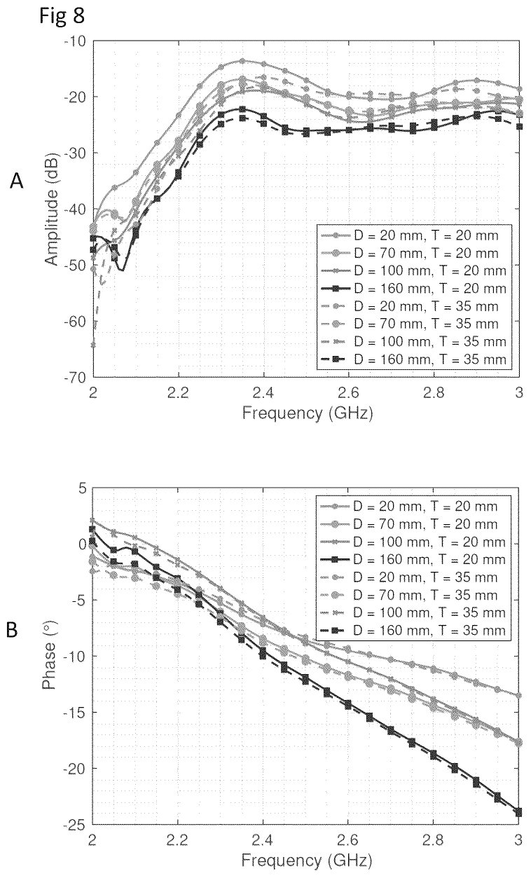

To analyze further, the variation of distance between the SRR sensors, which is increased from 20 mm to 250 mm, is investigated. FIGS. 8A and 8B shows the results of the amplitude and phase when the thickness of skin=2.5 mm, fat=20 and 35 mm, and muscle 50 mm. The variation in signal coupling due to the change in distance gradually decreases to about 8 dB when the SRR distance is increased. However, there is no significant discrepancy on phase variation over different thickness. It can be seen that the thickness does not strongly affect the phase of the transmission with respect to the fat layer. The SRRs demonstrate EM coupling but not EM propagating; therefore, no significant phase difference is observed. The amplitude of the signals is affected by the channel thickness.

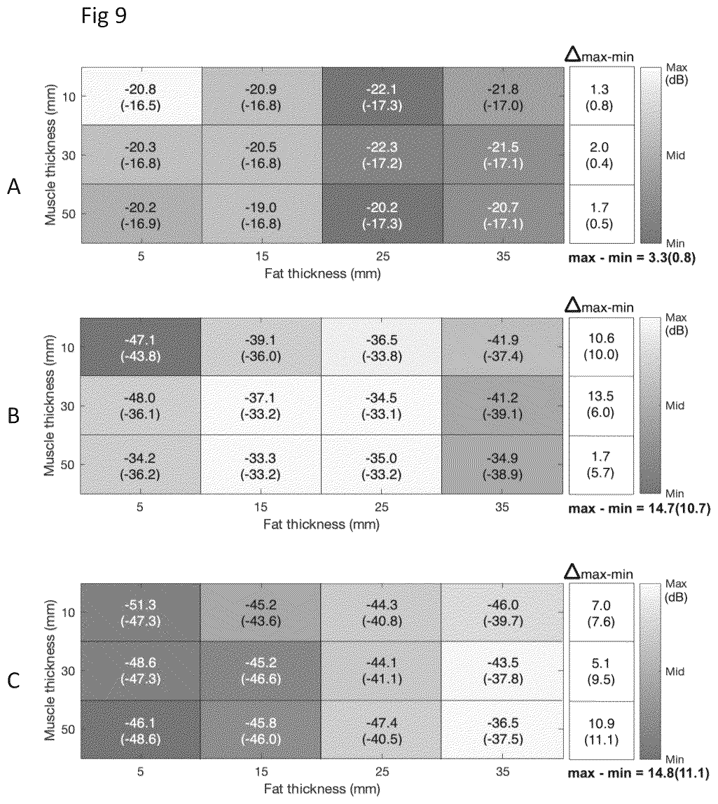

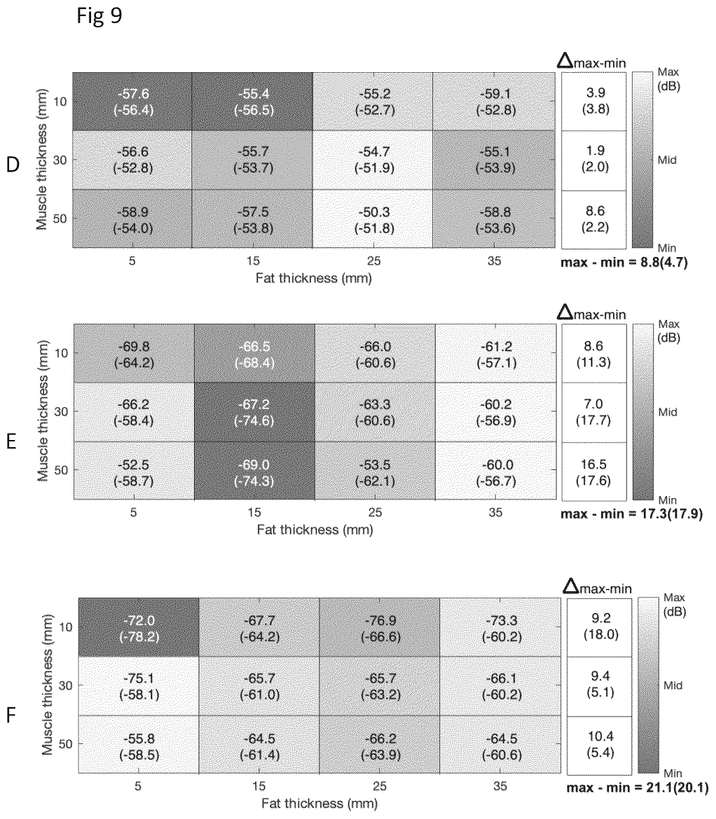

Next, FIG. 9 shows the results of the simulation and measurement of homogeneous layer tissues consisting of skin, fat, and muscle (organized from top to bottom). To characterize the signal loss of the multilayer tissue, the layered fat and muscle tissues were defined as a function of the distance between the two SRR sensors. As illustrated in FIGS. 9(a)-(f), the solid values correspond to the results of the measurements and the values inside the parentheses correspond to the results of the simulation. The color bar index indicate the color variation correspond to S21 result (in dB) of the measured and simulated (within parantheses) from minimum to maximum values.

From results shown in the FIG. 9(a), the observation of signal loss is between 19.0 dB and 22.3 dB for the 0 mm distance position. It shows the variability that the fat thickness produce signal loss at the fixed thickness of the muscle. In the 0 mm position, the received signal is significantly reduced by 2.1 dB at 25 mm fat-layered tissue case that includes skin and muscle variation. This can be explained by signal diffusion due to high attenuation and frequency dispersion in muscle tissue of the microwave signals. In addition, most of the reflection signal in the fat layer dissipates and penetrates to deeper tissue layers.

Furthermore, we observed that the influence of the increasing distance between Tx and Rx sensors as a function of the layered fat and muscle tissue does provide significant difference on signal loss as shown in FIG. 9(b). The first strong effect is observed at the boundary between thinner of fat and muscle, where the magnitude of signal loss dropped by up o −43.8 dB for simulation and −48.0 dB for measurement. The contrast between skin and fat as well as between fat and muscle being very high, strong reflections also occur at these two boundaries.

Another notable aspect that can be seen in FIG. 9(c) is the measured values slightly increased when 35 mm thick fat and 50 mm muscle tissue are used. The results show, for example, that when muscle is remaining constant in thicker layer it has an attenuation of 9.6 dB/cm in difference. This is in line with expectations mainly caused by EM signals due to their high permittivity and loss; they are significantly reduced before they reach the receiver. On the other hand, the effect of material absorption in these layers can be observed to be slightly lower in relation to the behavior of a strong reflection signal when looking at the thinner layers of fat tissue of all the figures. This is because the fat tissue layers used are much thinner than the corresponding penetration depths (117.1 mm at 2.45 GHz), and once the E-field is confined to the fat limit, it constantly attenuates.

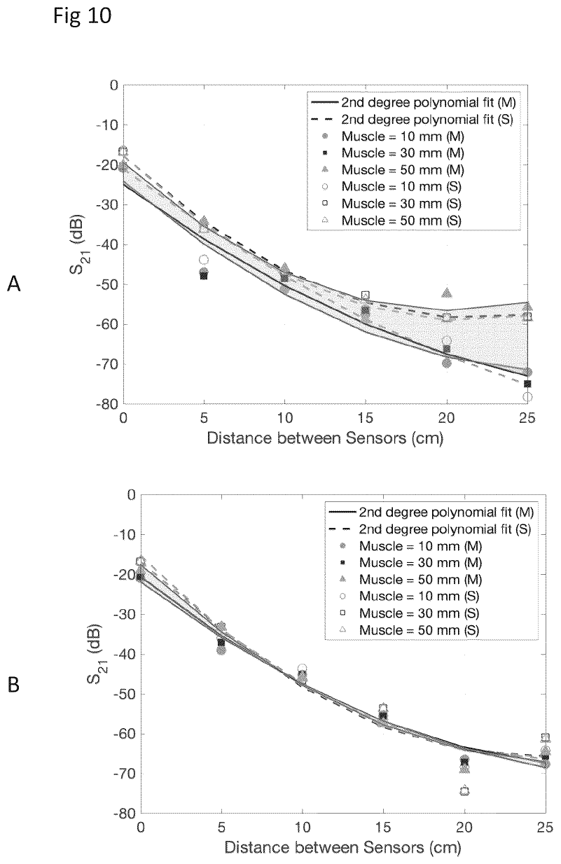

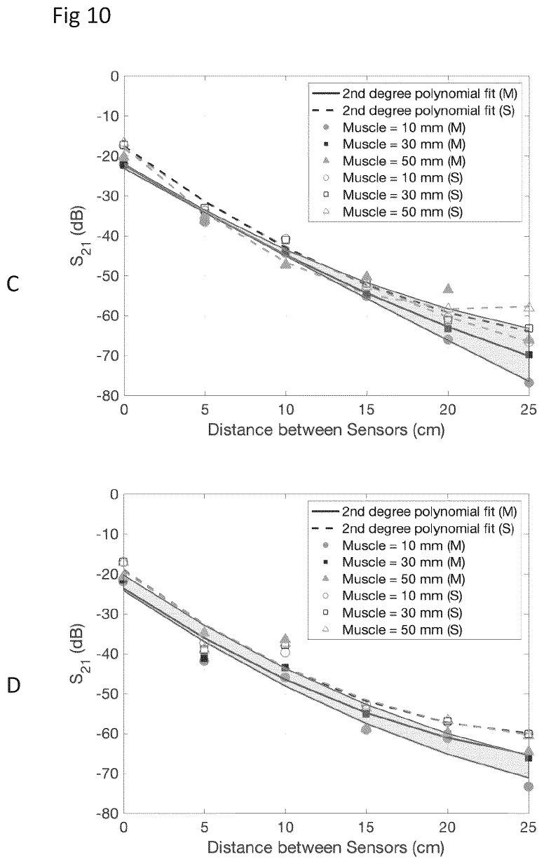

In FIG. 10A-10D, a comparison of signal loss is shown for different muscle thicknesses while the fat layer is fixed. The signal loss of 5 mm of fat thickness at a distance of 0 mm with a minimum muscle of 10 mm is 24.2 dB and approximately 20 dB with a maximum muscle of 50 mm. The signal loss increases from 72 dB to 54 dB for maximum and minimum muscle thickness at a distance of 25 cm. Meanwhile, we observed the signal loss of 35 mm of fat thickness having a smaller variation from 19 dB to 22.5 dB for maximum and minimum muscle thickness at 0 mm distance and between 68 dB and 64 dB for 25 cm distance. The high contrast in the dielectric properties between the muscle and the fat layer allows even thin muscle layers to act as good boundaries that confine signals within the fat layer. The results of this investigation provide more understanding of the signal loss and can be utilized to extend or evaluate tissue thickness apply a fixed sensor position.

In summary FIG. 10A-10D, the different fat thickness shows a very distinctive curve/pattern, which could be used to distinguish the thickness of underlying tissues. The standard deviation of the sensor is ±0.08, which shows that the data points are significantly different from each other.

Ex Vivo Experimental Set-Up and Measurements

We emulate human tissues using fresh porcine belly as commonly used in in-body imaging and power transfer systems. These tissues have layer structures that are complex and include skin, fat and muscle. Furthermore, these tissue EM properties are similar to human tissues. This tissue material, therefore, provides an ideal environment for human tissue emulation. The skin, fat and muscle porcine belly tissue were separated and finely minced with a meat mincer. The three-layered tissue structure, which contains skin, fat and muscle (top to bottom), is placed and arranged in a custom-made 3D printed plastic container and supported by a 5 mm-thick plate below the muscle layer.

The size of mold are 250 mm length, 60 mm width and 80 mm height. By varying the leveling plate underneath muscle layer, the thickness of the fat and muscle layer in exvivo tissue can be changed. On the other hand, a flexible, broadband, lightweight and multilayer flat carbon loaded laminate polyurethane (PU) foam based microwave absorber is used for experimental setup (FU-ML-120, Sahajanand Laser Technology Ltd, Gujarat, India). The dimension of microwave absorber are 600 mm length, 600 mm width and 120 mm height and reflectivity performance is −17 dB at 2.0 GHz.

The SRR sensors were attached to the surface of skin layers by using a stretchable strap to be sure that the sensor remains in good contact with skin and retains a constant pressure throughout the measurement. The sensors were then aligned as shown in FIG. 2(b) and connected to the Fieldfox Microwave Analyzer (N9918A). The measurement was conducted in the frequency range of 1-4 GHz and the resonance frequency of the sensor is optimized to be 2.35 GHz in a normal condition (skin=2.3 mm, fat=5 mm and muscle=10 mm). In order to investigate the signal loss, S₂₁ through the tissues, the distance between two SRR sensors was varied from 0 mm to 250 mm during the measurement. These distances were chosen based on the clinical US measurement adopted in previous studies.

Additionally, this ex-vivo model was examined to gauge the depth of penetration by analyzing the E-field distribution of the layered tissues. Inferences from the E-field distribution simulation were made to characterize the signal loss in the experimental setup and the results have been compared. The penetration depth provides good information for analysing sensor performance, especially E-field distribution in different tissues, and can be used for future work in clinical measurements.

The electromagnetic (EM) waves mainly propagate around the human body surface via diffusion. As an outcome, the human body, a high-loss dielectric medium, usually has huge impacts on the signal propagation. Additionally, as the human tissues contain a range of dielectric properties, a functional model of the body would need to be studied on the signal propagation. Specifically, when RF signals propagate from a high dielectric property medium (like skin or muscle) to low dielectric property medium (like fat), it bends away from the direction perpendicular to the interface between the materials. This means that any signal that is reflected into the body has to travel across multiple centimeters (cm) of multilayer tissue and face multiple reflections before it can exit to the air. Thus, signal propagation in each layer is assumed to be linear, but across layers, it can change to multiple directions. To validate the effect of fat and muscle layer variation, the thickness of skin (2.5 mm) layer remains fixed; meanwhile, the variation of fat thickness is adjusted from 5 mm to 35 mm by 10 mm steps. Furthermore, the variation of muscle thickness from 10 mm to 50 mm by 20 mm steps has also been considered. It is necessary to calculate and sum up the maximum and minimum difference of magnitude of S₂₁, |S₂₁|, required to propagate across each model layer. To calculate the signal loss, skin and muscle are considered to remain constant and the maximum and minimum signal loss are reckoned by varying fat layer thickness. Therefore, the different signal loss, S₂₁ (in dB), can be calculated by:

Δ_{signalloss}=Max−Min (1)

where Δ_{signalloss} is the signal loss, S₂₁ (dB) and Max-Min is the difference between maximum and minimum signal at any variation of fat and muscle thicknesses.

This will provide a basis for the comparison of approximations that can be used from human tissue model for reflectivity and refractivity, and their suitability for integration into larger EM models.

E-Field Distribution Analysis

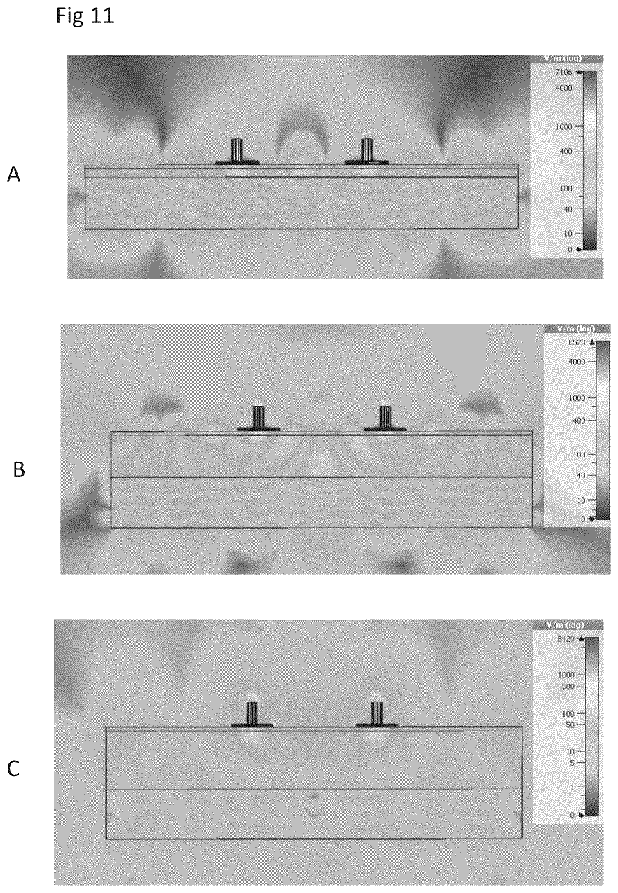

In addition to the described attenuation of the signal due to the difference in dielectric properties, the distribution of E-field is presented when propagating from one layer to another. FIG. 11A-11C shows a two dimensional (2D) E-field distribution by varying the fat thickness.

We considered three experimental scenarios to inspect the E-field distribution between Tx and Rx sensor at a fixed distance of 100 mm:
1) Scenario 1: Minimum thickness of fat layer as 5 mm to represent a thinner condition.
2) Scenario 2: An average thickness of fat layer as 25 mm to represent a normal condition.
3) Scenario 3: Maximum thickness of fat layer as 35 mm to represent a high-fat condition.

FIG. 11A explains the E-field distributions of the thinner condition in the fat tissues, which results in higher surface coupling and leakage of the signal. The E-field highly leaks outside skin layer from the fat layer to the surrounding free space. In this case, the consideration of multiple reflections can occur between the surface layer and fat tissue boundaries. Therefore, the layer of fat showed enlargement of the E-field close to the Rx sensor with the prominence of mismatching on the Tx sensor.

In FIG. 11B, we observe that increasing the fat layer enables the E-field to propagate further through the muscle tissues. It is thus observed that attenuated signal in this layer is small compared to scenario 1 in the muscle tissue layers of this condition. It is important to note that there are two significant contributions; (i) the loss of absorption due to the material properties and (ii) the loss of the reflection signal as it propagates through multiple tissue layers.

For the arrangement of scenario 3 (FIG. 11C) where maximum fat tissue thickness is 35 mm, the signal is continuously attenuated for thicker fat as the result sets are very similar to scenario 2. Therefore, we observe that while increasing the fat layer in between 25 mm and 35 mm, there is no significant change in E-field distribution through fat-muscle boundaries.

The transmitted RF signal is constantly attenuated while passing through the fat tissue where the attenuation depends on the thickness of fat. Hence, the reflected signal from the next tissue declines even further and is causing the exponential fading of signal, especially from the beginning of the muscle tissue.

In summary, we observe two types of eigenmodes, namely, the bound states and the free state. The former are the modes bounded in the fat layer and they trap the signal mainly in the layer between skin and muscle. The latter are modes that trap the signal of the exterior mode, which are not bound to the layered fat but are flowing in the open regions.

Penetration Depth Assessment

Penetration depth was observed by examining the same experimental scenario from the previous section. The examination was done from the simulated E-field and the results were correlated. The defined E-field position was thus got from the axis E_{z}. Thus, the E-field distribution was measured perpendicularly to the Tx sensor plane along the E_{z} axis. The starting position of the E_{z} axis was taken into account at the maximum E-field strength at the sensor interface and the surface of the skin.

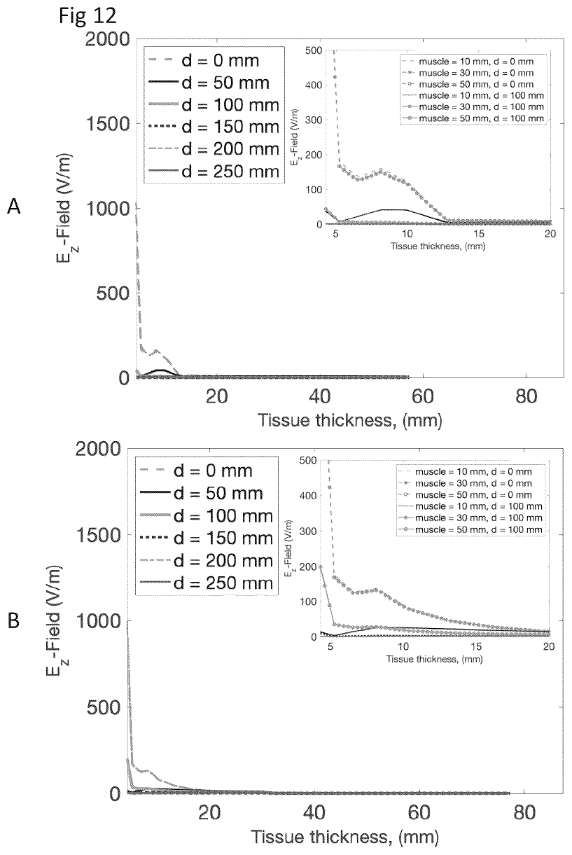

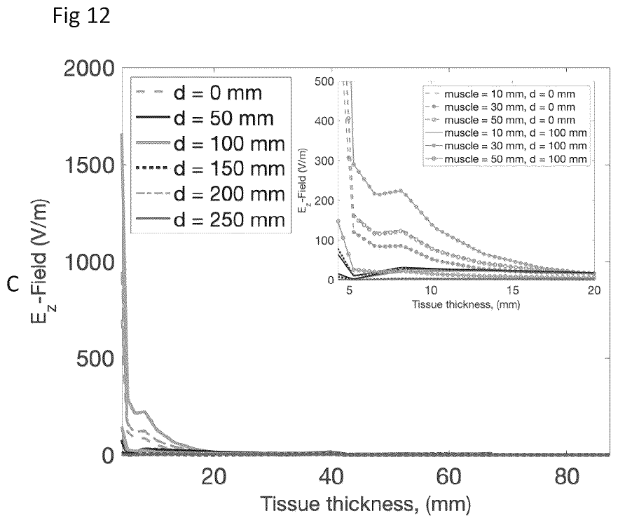

FIG. 12A-12C show that similarity performance at penetration depth expands throughout the fat layer (2.5 mm to 10 mm on average on the tissue thickness axis) and the intensity of the E-field rises at the skin-fat boundary. Considering the tissue thickness information, when a thicker fat layer is present, the penetration depth was discovered to be greater. Thus, the thickness of the fat layer is observed to have a great impact on the distribution of the E-field. Because fat tissue has inherently low water content, its dielectric characteristics show very low frequency dispersion. In addition, the extent of the E-field represents the percentage increase in the magnitude of the E-field in the fat layer. Using the following equation, it is calculated:

$${E}_{extend} = ( \frac{Max}{Min} - 1 ) \times 100 \% \qquad ( 2 )$$

On average, 200 V/m (44.5%) of the E-field intensity increases on a boundary skin-fat and decreases into the next layer. The depth was gradually decreased once the E-field arrives at an average fat thickness of 10 mm. Another notable aspect to be observed in FIG. 12 is that the E-field initiates to be interrupted approximately 60 mm at the minimum tissue thickness (scenario 1) and extends more than 80 mm for maximum tissue thicknesses (scenario 3).

Looking at the multilayer tissue thickness composition of all FIG. 12, it can be seen that at the boundary fat-muscle the material absorption is caused. Therefore, the impact of dielectric properties that are highly different between the muscle and the fat layer which strongly affects the penetration depth. According to these results, the signal will fluctuate at this depth because of the appearance of reflected signals.

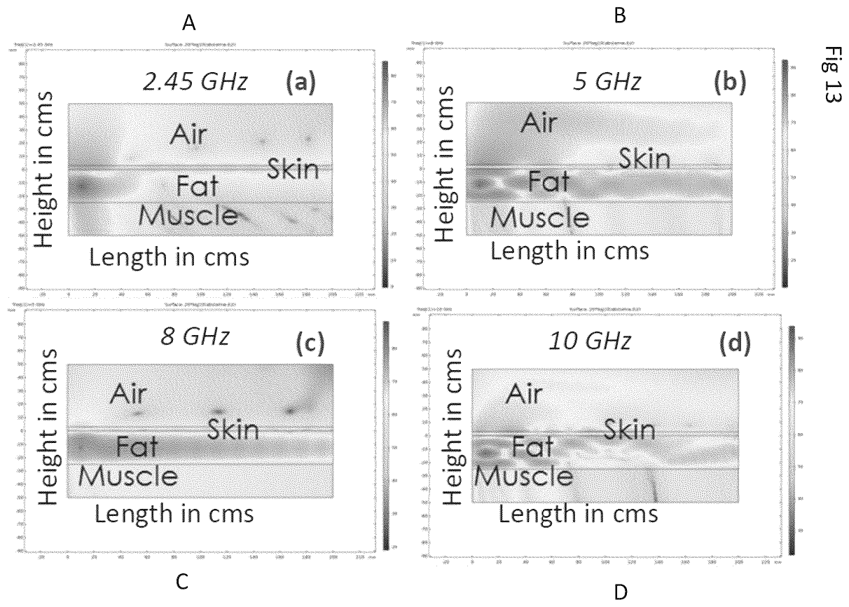

The system and method according to the invention is useful in the quantification of muscle mass at any anatomical location of human body. It uses microwave signals which are selective towards human tissue properties. Since the passage of microwave signal is affected by various human tissues differently it gives the perfect opportunity to quantify any specific or group of tissues that microwave passes through. The microwave signal generator is configured to generate different signal frequencies enabling the signal confinement in various tissues as shown in the FIG. 13. FIG. 13 shows microwave signal confinement in different tissues with respect to the frequency of operation; a) Skin is targeted at 2.45 GHz. b) Signal confines in fat at 5 GHz. c) Signal confines to fat an muscle at 8 GHz and d) Signal spreads into muscle at 10 GHz. Repetition of signal propagation through the biological tissue provides for accurate quantification of subdermal tissues such as fat and muscle which is otherwise extremely difficult.

The person skilled in the art realizes that the present disclosure is not limited to the preferred embodiments described above. The person skilled in the art further realizes that modifications and variations are possible within the scope of the appended claims. Additionally, variations within the scope of the appended claims can be understood and effected by the skilled person in practicing the claimed disclosure, from a study of the drawings, the disclosure, and the appended claims.

1. A sensor arrangement comprising a support substrate and at least two microwave antennas fixedly arranged in or on said support substrate, each microwave antenna being separated from all other microwave antennas by a band-stop structure configured to cancel direct coupling between the microwave antennas, said sensor arrangement further comprising microwave signal transmission paths configured to transmit microwave signals to or from said microwave antennas.

2. The sensor arrangement according to claim 1, wherein the microwave antennas are fixedly arranged 20-160 mm apart.

3. The sensor arrangement according to claim 1, wherein said microwave antennas are split ring resonators.

4. The sensor arrangement according to claim 1, further comprising one or more microwave signal contacts connected to the transmission paths to the microwave antennas.

5. The sensor arrangement according to claim 4, comprising at least one microwave switch arranged in a transmission path between a microwave signal contact and two or more microwave antennas.

6. The sensor arrangement according to claim 1, wherein at least three microwave antennas are fixedly arranged in or on said support substrate.

7. The sensor arrangement according to claim 1, wherein the support substrate is flexible.

8. The sensor arrangement according to claim 1, wherein the support substrate has an adhesive coating for fixing the sensor arrangement on the skin of a subject.

9. A system comprising a sensor arrangement according to claim 1; a tuneable microwave signal generator configured to generate and transmit a microwave signal to a first microwave antenna in said sensor arrangement; and a signal processing unit arranged to receive the transmitted microwave signal from at least one second microwave antenna in the sensor arrangement.

10. The system according to claim 9, wherein the signal processing unit is configured to analyse a change in the transmitted microwave signal between said first microwave antenna and said at least one second microwave antenna.

11. The system according to claim 9, wherein the change in said microwave signal is a change in amplitude and/or a phase delay.

12. The system according to claim 9, wherein the tuneable microwave signal generator generates microwave signals in a frequency range of 2.45 GHz to 10 GHz.

13. The system according to claim 9 which is configured as a system for non-invasive assessment of a property of subdermal tissue in a subject, wherein the signal processing unit is configured to correlate a change in the transmitted microwave signal between the first microwave antenna and the at least one second microwave antenna with properties of subdermal tissue in the subject.

14. The system according to claim 13, wherein the sensor arrangement comprises at least three microwave antennas and the signal processing unit is
arranged to receive said microwave signal from at least two second microwave antennas in said sensor arrangement; and
configured to correlate a change in the transmitted microwave signal between the first microwave antenna and each of the at least two second microwave antennas with the property of subdermal tissue in a subject.

15. The system according to claim 13, wherein the property of subdermal tissue is selected from the group consisting of fat content of muscle tissue, thickness of muscle tissue, thickness of fat tissue.

16. A method for non-invasive assessment of a property of subdermal tissue in a subject comprising the steps
S1. Providing a first and at least one second microwave antennas in connection with the skin of said subject, each microwave antenna being separated from all other microwave antennas by band-stop structures configured to cancel direct coupling between the microwave antennas and provided at a fixed distance from all other microwave antennas;
S2. Transmitting a microwave signal from the first microwave antenna;
S3. Receiving the microwave signal at the at least one second microwave antenna after propagation through subdermal tissue of the subject;
S4. Optionally repeating steps S2 and S3 with a microwave signal of a different frequency; and
S5. Correlating a change in the transmitted microwave signal or signals between the first microwave antenna and the at least one second microwave antenna with properties of subdermal tissue in the subject.

17. The method according to claim 16, comprising the steps
S1. Providing a first and at least two second microwave antennas in connection with the skin of said subject, each microwave antenna being separated from all other microwave antennas by band-stop structures configured to cancel direct coupling between the microwave antennas and provided at a fixed distance from all other microwave antennas;
S2. Transmitting a microwave signal from the first microwave antenna;
S3. Receiving the microwave signal at each of the at least two second microwave antennas after propagation through subdermal tissue of the subject;
S4. Optionally repeating steps S2 and S3 with a microwave signal of a different frequency; and
S5. Correlating a change in the transmitted microwave signal or signals between the first microwave antenna and each of the at least two second microwave antennas with properties of subdermal tissue in the subject.

18. The method according to claim 16, wherein the change in the transmitted microwave signal is a change in amplitude and/or a phase delay.

19. The method according to claim 16, wherein steps S2 and S3 are repeated at least four times with microwave signals of frequency 2.45; 5, 8, and 10 GHz.

20. The method according to claim 16, wherein the property subdermal tissue is selected from the group consisting of fat content of muscle tissue, thickness of muscle tissue, and thickness of fat tissue.

21. The method according to claim 16, wherein the microwave antennas are provided by a sensor arrangement.

22. Use of a sensor arrangement according to claim 1 in a method for non-invasive assessment of a property of subdermal tissue in a subject comprising the steps
S1. Providing a first and at least one second microwave antennas in connection with the skin of said subject, each microwave antenna being separated from all other microwave antennas by band-stop structures configured to cancel direct coupling between the microwave antennas and provided at a fixed distance from all other microwave antennas;
S2. Transmitting a microwave signal from the first microwave antenna;
S3. Receiving the microwave signal at the at least one second microwave antenna after propagation through subdermal tissue of the subject;
S4. Optionally repeating steps S2 and S3 with a microwave signal of a different frequency; and S5. Correlating a change in the transmitted microwave signal or signals between the first microwave antenna and the at least one second microwave antenna with properties of subdermal tissue in the subject.

23. Use of a system according to claim 9 in a method for non-invasive assessment of a property of subdermal tissue in a subject comprising the steps
S1. Providing a first and at least one second microwave antennas in connection with the skin of said subject, each microwave antenna being separated from all other microwave antennas by band-stop structures configured to cancel direct coupling between the microwave antennas and provided at a fixed distance from all other microwave antennas;
S2. Transmitting a microwave signal from the first microwave antenna;
S3. Receiving the microwave signal at the at least one second microwave antenna after propagation through subdermal tissue of the subject;
S4. Optionally repeating steps S2 and S3 with a microwave signal of a different frequency; and
S5. Correlating a change in the transmitted microwave signal or signals between the first microwave antenna and the at least one second microwave antenna with properties of subdermal tissue in the subject.
